(19) United States
(12) Patent Application Publication
(10) Pub. No.: US 2008/0132417 A1
(43) Pub. Date: Jun. 5, 2008

(54) Molecular Basis For The Identification Of Chemotherapy Resistance In Human Tumors And The Treatment Thereof

(75) Inventors: Donald J. Buchsbaum, Alabaster, AL (US); Martin R. Johnson, Birmingham, AL (US); James A. Bonner, Birmingham, AL (US); Kevin P. Raisch, Trussville, AL (US)

Correspondence Address: FISH & RICHARDSON P.C., P.O BOX 1022, Minneapolis, MN, 55440-1022, US

(73) Assignee: THE UAB RESEARCH FOUNDATION, Birmingham, AL (US)

(21) Appl. No.: 11/719,017

(22) PCT Filed: Nov. 16, 2005
(86) PCT No.: PCT/US05/41618

§ 371 Date: Jul. 20, 2007

Related U.S. Application Data
(60) Provisional application No. 60/628,510, filed on Nov. 16, 2004.

(51) Int. Cl. C40B 20/02; G06F 17/00; C12Q 1/68; C40B 40/06; C40B 20/00

(52) U.S. Cl. 506/3; 707/104.1; 435/6; 506/16; 506/2; 707/E17.009

(57) ABSTRACT

Provided are compositions and methods for determining whether a tumor in a subject is sensitive or resistant to a chemotherapeutic agent. Further provided are methods and compositions for identifying a chemotherapeutic agent to which a subject's tumor is resistant or sensitive. Methods and compositions for selecting a chemotherapeutic agent for treating a subject with a tumor are also provided.

This application claims priority to U.S. provisional application No. 60/628,510 filed on Nov. 16, 2004. The aforementioned application is herein incorporated by this reference in its entirety.

### BACKGROUND OF THE INVENTION

This invention relates generally to the field of cancer chemotherapy. Specifically, the invention relates to methods of determining whether a tumor in a subject is resistant or sensitive to a selected chemotherapeutic agent. The invention is further related to methods of selecting a chemotherapeutic agent for treating a tumor and monitoring the effectiveness of therapy.

Treating various types of cancer with certain drugs often leads to disparate results and prognoses among patients. Some subjects experience successful treatment with chemotherapy and go into remission, while others do not. Other subjects find that a combination or cocktail of drugs works better than one drug alone.

Until the discovery of the present invention, it has generally been difficult to predict and/or determine with good success which drug or combination of drugs works better for certain cancers, and more importantly, for particular subjects having that cancer. Therefore, a need exists to select a chemotherapeutic agent or agents for particular types of cancer and tissue types. A further need exists to individually tailor chemotherapy to maximize the benefits of treatment.

### SUMMARY OF THE INVENTION

The invention provides a set of informative genes whose expression correlates with a chemosensitive class or a chemoresistant class across samples, wherein the samples are either sensitive or resistant to a compound (e.g., a chemotherapeutic agent). A gene whose expression correlates with either the chemosensitive class or chemoresistant class more strongly than expected by chance is an informative gene.

Thus, provided herein are compositions and methods for determining whether a tumor in a subject is sensitive or resistant to a chemotherapeutic agent. Further provided is a method of identifying a chemotherapeutic agent to which a subject's tumor is sensitive or to which the subject's tumor is resistant. Methods of selecting a chemotherapeutic agent for treating a subject with a tumor are also provided. Also provided herein are arrays and kits for selecting a chemotherapeutic agent to treat a tumor in a subject. Methods for determining whether a tumor has acquired resistance to a chemotherapeutic agent are also provided. Also provided herein are arrays, computer-readable media, and computer systems used in detecting and analyzing one or more of the informative genes.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 shows reducing the CPT-11 resistance of SW948CPTH cells by treatment with IMC-C225 (C225) or siXRCC1. Cells were treated with C225, Oligofectamine (Oligo, vehicle control for siXRCC1) or siXRCC1 for 24 h prior to the addition of CPT-11. The CPT-11 IC₅₀ for the C225 treated cells was decreased by 26% as compared to the untreated group (p<0.01). The CPT-11 IC₅₀ for the siXRCC1 treated cells was decreased by 25% as compared to the Oligofectamine group (p<0.05). The data are the average ±SE of two to four independent experiments performed in quadruplicate. p-values were determined by Student's t-test.

FIG. 2 shows XRCC1 expression in SW948CPTH and SW948 cells after treatment with C225. C225 was added to the cell culture medium at time 0, and the cells were incubated at 37° C. for 72 h. Cell lysates were collected at 0, 4, 24, 48 and 72 h. Immunoblot analyses indicate a reduction in XRCC1 protein levels at 24-72 h in SW948CPTH cells but no observable change in XRCC1 protein levels in SW948 cells.

FIG. 3 shows dose schema for development of CPT-11 resistance. FIG. 1A shows the generation of SW948CPTH cell line. FIG. 1B shows the generation of SW948CPTL cell line. Up arrows indicate addition of CPT-11 to the culture medium, and down arrows indicate removal of CPT-11 from the culture medium. Days indicate the time at which CPT-11 concentrations were changed.

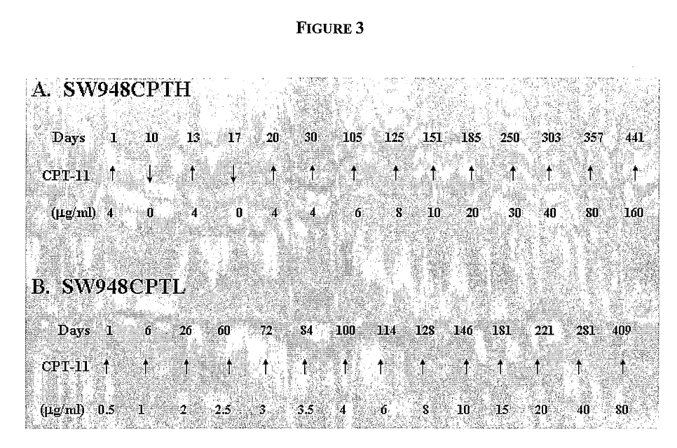

FIG. 4 shows immunoblots of EGFR and ErbB-2 protein from SW948, SW948CPTH and SW948CPTL cell lysates. 20 μg protein was loaded per lane. The blots were probed with anti-EGFR (Sigma) or ErbB-2 (Santa Cruz), followed by anti-mouse-HRP or anti-rabbit-HRP, respectively. The blots were developed with chemiluminescence.

FIG. 5 shows relative levels of p-glycoprotein (p-gp) and breast cancer resistant protein (BCRP) in SW948, SW948CPTH, and SW948CPTL cells. SW948CPTH and SW948CPTL cells were cultured in medium without CPT-11 for 21 or 7 days, respectively. Cells were washed with phosphate buffered saline, scraped, and aliquoted into tubes. Cells were incubated with antibodies against p-gp or BCRP for 30 min at 4° C., followed by FITC-conjugated goat anti-mouse antibody. Fluorescence intensity of the cells was monitored and analyzed by flow cytometry. Data are shown as mean fluorescence intensity ±SD (n=3-6).

FIG. 6 shows effects of verapamil on the intra-cellular accumulation of CPT-11 in SW948, SW948CPTH, and SW948CPTL cells. Cells were incubated with CPT-11 [160 μM] for 2 h at 37° C. in the presence or absence of 10 μM verapamil. Cells were washed, trypsinized, and counted. Cell pellets were lysed with water and sonicated. The amounts of CPT-11 in the supernatant harvested from the samples were determined by HPLC. The values of CPT-11 were calculated as μmoles/106 cells. Data are expressed as mean ±SD (n=3).

FIG. 7 shows immunoblot of XRCC1 protein from SW948, SW948CPTH and SW948CPTL cell lysates. 20 μg protein was loaded per lane. The blot was probed with anti-XRCC1 (NeoMarkers), followed by anti-mouse-HRP. The blot was developed with chemiluminescence and scanned using UN-SCAN-IT software. The data from 2-3 independent blots was normalized to 100% in the SW948 cells and presented as the average ±SD.

FIG. 8 shows immunoblot of XRCC1 and actin proteins from SW948, SW948CPTH and SW948CPTL cells exposed to cetuximab (IMC-C225) for 1 h, 4 h, 24 h or 48 h. Cell lysates were collected at each time point and 20 μg protein was loaded per lane. The blot was probed with anti-XRCC1 (NeoMarkers) or anti-actin (Santa Cruz), followed by anti-mouse-HRP or anti-goat-HRP, respectively. The blots were developed with chemiluminescence and scanned using UN-SCAN-IT software. The data was normalized for actin expression at 100% for each untreated cell line.

### DETAILED DESCRIPTION OF THE EMBODIMENTS OF THE INVENTION

The present invention may be understood more readily by reference to the following detailed description of preferred embodiments of the invention and the Examples included therein.

Before the present compounds, compositions, articles, devices, and/or methods are disclosed and described, it is to be understood that the aspects are not limited to specific methods or specific chemotherapeutic agents, as such may, of course, vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “an expression profile” can include more than one expression profile; reference to “a chemotherapeutic agent” includes mixtures of two or more such agents, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

“Optional” or “optionally” means that the subsequently described event or circumstance can or cannot occur, and that the description includes instances where the event or circumstance occurs and instances where it does not. For example, the phrase “optionally converting the mRNA to cDNA” means that the mRNA may be converted to cDNA or the mRNA may not be converted to cDNA and that the description includes both mRNA and cDNA.

Cancer is a disease for which several classes or types exist, many requiring different treatments. Thus, cancer is not a single disease but rather a family of disorders arising from distinct cell types by distinct pathological mechanisms. The challenge of cancer treatment has been to target specific therapies to particular tumor types, to maximize effectiveness and to minimize toxicity. Improvements in cancer classification have thus been central to advances in cancer treatment. Additionally, the outcome of chemotherapy using a particular chemotherapeutic agent or set of chemotherapeutic agents can be predicted, or the efficacy determined, based on the analysis of gene expression data as disclosed herein.

Provided herein are compositions and methods that allow a person of skill to determine whether a tumor in a subject is sensitive or resistant to a selected chemotherapeutic agent. Further provided is a method of identifying a chemotherapeutic agent to which a subject's tumor is resistant. Also provided herein is a method of identifying a chemotherapeutic agent to which a subject's tumor is sensitive. Methods of selecting a chemotherapeutic agent for treating a subject with a tumor are also provided. Also provided herein is a kit for selecting a chemotherapeutic agent to treat a tumor in a subject. Further provided are tools, such as arrays, computer-readable media, and computer systems, for analyzing gene expression related to chemoresistance or chemosensitivity. Methods for determining whether a tumor has acquired resistance to a chemotherapeutic agent are also provided.

Disclosed are methods and compositions for classifying a biological sample, for example, a cell from a tumor, using gene expression levels in the sample. The methods involve assessing the sample for the level of gene expression for at least one gene and classifying the sample as being resistant or sensitive to a chemotherapeutic agent by determining the similarity or dissimilarity of the gene expression profile of the sample to a reference gene expression profile of one or more genes, wherein the reference gene expression profile correlates with resistance or sensitivity to a selected chemotherapeutic agent.

Thus, provided herein is a method of determining resistance of a tumor cell to a chemotherapeutic agent, comprising: a) determining an expression profile of one or more DNA repair genes from the tumor cell; and b) comparing the profile of step (a) to a reference expression profile, wherein the reference profile correlates with resistance to the chemotherapeutic agent, a similar expression profile of genes from the tumor cell compared to the reference expression profile indicating resistance of the tumor cell to the chemotherapeutic agent. DNA repair genes include, but are not limited to, the genes listed in Tables 1 and 2. More specifically, the DNA repair genes include XRCC1, MGMT, RPA2, CCNH, ERCC1, MUS81, XRCC5 (Ku80), DUT, REV1L (REV1), UBE2B (RAD6B), RECQL (RECQ1), ERCC2 (XPD), ERCC6, PMS2, PCNA, TREX1 (DNase III) and mutL homolog 3 (MLH3). The DNA repair gene interest group sponsored by the National Institutes of Health has produced an updated table of 148 Human DNA repair genes (see DNA Repair Special Interest Group, and Wood et al. (Science (2001) 291, 1284, which is incorporated herein in its entirety by this reference for the purpose of listing the DNA repair genes). The genes listed in Tables 1 and 2 are divided into subcategories based on their known mechanism of action/activity.

In addition to the DNA repair genes, the invention includes a method of determining resistance of a tumor cell to a chemotherapeutic agent, comprising: a) determining an expression profile of one or more genes selected from the group of genes consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor, beta receptor III (TGFBR3) from the tumor cell; and b) comparing the profile of step (a) to a reference expression profile, wherein the reference profile correlates with resistance to the chemotherapeutic agent, a similar expression profile of genes from the tumor cell compared to the reference expression profile indicating resistance of the tumor cell to the chemotherapeutic agent.

TABLE 1
List of Human DNA repair genes up (+) or down (−) regulated, as
defined by fold change, in SW948CPTH cells as compared to the parental
cell line, SW948. The genes are categorized by mechanism of activity.

| | | Fold | | |
|---|---|---|---|---|
| Gene Name | | change vs | Chromosome | Accession |
| (synonyms) | Activity | SW948 | location | number |
| | Base excision repair (BER) | | | |
| | factors | | | |
| XRCC1 | Ligase accessory factor | +1.41 | 19q13.31 | NM_006297 |
| | Direct reversal of damage | | | |
| MGMT | O6-meG alkyltransferase | −1.25 | 10q26.3 | NM_002412 |
| | Nucleotide excision repair | | | |
| | (NER) | | | |
| RPA2 | RPA1, RPA2, RPA3 | +1.42 | 1p35.3 | NM_002946 |
| CCNH | CDK7, CCNH, MNAT1 | +1.32 | 5q14.3 | NM_001239 |
| ERCC1 | 5′ incision subunit | +1.46 | 19q13.32 | NM_001983 |
| | Homologous recombination | | | |
| MUS81 | A structure-specific DNA | −1.16 | 11q13.1 | NM_025128 |
| | nuclease | | | |
| | Non-homologous end-joining | | | |
| XRCC5 (Ku80) | Ku70, Ku80 | +1.23 | 2q35 | NM_021141 |
| | Modulation of nucleotide | | | |
| | pools | | | |
| DUT | dUTPase | +1.69 | 15q21.1 | NM_001948 |
| | DNA polymerases (catalytic | | | |
| | subunits) | | | |
| REV1L(REV1) | dCMP transferase | +1.62 | 2q11.2 | NM_016316 |
| | Rad6 pathway | | | |
| UBE2B | Ubiquitin-conjugating enzyme | +1.47 | 5q31.1 | NM_003337 |
| (RAD6B) | | | | |
| | Other identified genes with a | | | |
| | suspected DNA repair | | | |
| | function | | | |
| RECQL | DNA helicase | +1.33 | 12p12.1 | NM_002907 |
| (RECQ1) | | | | |

TABLE 2
List of Human DNA repair genes up (+) or down (−) regulated, as
defined by fold change, in SW948CPTL cells as compared to the parental
cell line, SW948. The genes are categorized by mechanism of activity.

| | | Fold | | |
|---|---|---|---|---|
| Gene Name | | change | Chromosome | Accession |
| (synonyms) | Activity | vs SW948 | location | number |
| | Mismatch excision repair | | | |
| | (MMR) | | | |
| PMS2 | MLH1, PMS2 | −1.41 | 7p22.1 | NM_000535 |
| | Modulation of nucleotide | | | |
| | pools | | | |
| DUT | dUTPase | +1.35 | 15q21.1 | NM_001948 |
| | DNA polymerases | | | |
| | (catalytic subunits) | | | |
| PCNA | Sliding clamp for pol delta and | −1.72 | 20p12.3 | NM_002592 |
| | pol epsilon | | | |
| | Editing and processing | | | |
| | nucleases | | | |
| TREX1 (DNase | 3′ exonuclease | +1.60 | 3p21.3-p21.2 | NM_016381 |
| III) | | | | |

Examples of chemotherapeutic agents include, but are not limited to, camptothecin (CPT); derivatives of CPT, for example, CPT-11 and 10-OH-CPT; and metabolites of CPT-11, for example, SN38. Examples of other chemotherapeutic agents with similar expression profiles include, but are not limited to, actinomycin D, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

As used herein, “resistance of a tumor cell to a chemotherapeutic agent” means that a tumor cell, when contacted with a chemotherapeutic agent at a concentration or dosage described in the art as generally therapeutically effective, will not stop growing and dividing.

An expression profile of one or more genes can be prepared from a biological sample, for example, cells from a tumor. An expression profile can list the expression values of mRNA production for each of a plurality of genes found in the cell population sampled. Therefore, provided is an expression profile of a plurality of genes from tumor cells of a cell line known to be sensitive to a selected chemotherapeutic agent. For example, cells from the human colon cancer cell line SW948 are known to be sensitive to CPT-11, a chemotherapeutic agent. A gene expression value of 1.00 can be assigned to each gene of the SW948 (sensitive) sample.

Further provided is a reference expression profile. As used herein, a “reference expression profile” is an expression profile that lists gene expression values for genes from cells of a cell line known to be resistant to a selected chemotherapeutic agent. For example, cell line SW948CPTH is a cell line resistant to CPT-11 that emerged after cells from the parent (sensitive) cell line SW948 were initially exposed to high concentrations of CPT-11 followed by the gradually increasing concentrations of CPT-11. Cell line SW948CPTL is a cell line resistant to CPT-11 that emerged after cells from the parent (sensitive) cell line SW948 were initially exposed to low concentrations of CPT-11 followed by the gradually increasing concentrations of CPT-11. The gene expression values for the resistant samples are calculated relative to the corresponding values of the sensitive SW948 cell line, which was assigned a normalized value of 1.00 for each gene. A value above 1 means that gene expression is up-regulated, and a value below 1 means gene expression is down-regulated. An isolated cell and a stable cell line that possess resistance to CPT or a derivative or a metabolite thereof and resistance to at least one additional chemotherapeutic agent is taught in PCT/US04/29696, filed Sep. 10, 2004, the contents of which are incorporated by reference herein in their entirety.

Gene expression can be assessed by a variety of methods including microarray methods like microarray real-time quantitative PCR (low density array analysis). Specifically, as taught in Example 2 below, gene expression can be determined using a Micro-Fluid Card™, manufactured by Applied Biosystems (Foster City, Calif.).

A person of skill can obtain a biological sample, for example, cells from a tumor in a subject, and prepare an expression profile of the genes from the sample. For example, an expression profile for cells from a subject's tumor can be prepared, listing the expression values of one or more of the selected genes.

Provided in Tables 1, 2, and 3 are the GenBank Accession Nos. for the human mRNA sequences and the GenBank Accession Nos. for the human protein sequences. The nucleic acid sequences and protein sequences provided under the GenBank Accession Nos. mentioned herein are hereby incorporated in their entireties by this reference. Fragments of the sequences provided under the GenBank Accession Nos. set forth herein are also provided by this invention and incorporated in their entirety by this reference. Furthermore, all of the information set forth under the GenBank Accession Nos. and all of the information accessible via the GenBank Accession Nos., for example, Entrez Gene information, is hereby incorporated in its entirety by this reference. One of skill in the art would know that the nucleotide sequences and the known functions of the genes provided under the GenBank Accession Nos. set forth herein can be readily obtained from the National Center for Biotechnology Information at the National Library of Medicine (http://www.ncbi.nlm.nih.gov/entrez/query.fcgi?db=nucleotide). Similarly, the protein sequences set forth herein can be readily obtained from the National Center for Biotechnology Information at the National Library of Medicine (http://www.ncbi.nlm.nih.gov/entrez/query.fcgi?db=protein).

After an expression profile of one or more genes from a subject's tumor cells is determined, a person of skill can compare the expression profile to a reference expression profile of matching genes to determine whether the expression profile of the subject's tumor cells is similar to the reference expression profile that correlates with resistance to a chemotherapeutic agent. If the expression profile of the cells from a subject's tumor is similar to the reference expression profile, the subject's tumor is determined to be resistant to the chemotherapeutic agent, for example, CPT-11.

For example, a person of skill can prepare an expression profile of one or more DNA repair genes or other genes taught herein from a subject's tumor cells and compare the expression profile to a reference expression profile of matching genes to determine whether the expression profile of the subject's tumor cells is similar to the reference expression profile which correlates with resistance to a chemotherapeutic agent. If the expression profile of the DNA repair genes from the cells from a subject's tumor is similar to the reference expression profile, the subject's tumor is determined to be resistant to the chemotherapeutic agent, for example, CPT-11.

In another aspect, a person of skill can prepare an expression profile of one or more other genes taught herein from a subject's tumor cells and compare the expression profile to a reference expression profile of matching genes to determine whether the expression profile of the subject's tumor cells is similar to the reference expression profile which correlates with resistance to a chemotherapeutic agent. If the expression profile of these genes from the cells from a subject's tumor is similar to the reference expression profile, the subject's tumor is determined to be resistant to the chemotherapeutic agent, for example, CPT-11.

As used herein, “similar” means “resembling though not necessarily completely identical.” Thus, a subject's expression profile can be similar to a reference expression profile when, for example, the subject's expression profile contains all of the genes listed in Tables 1-3 and all of those genes are up- or down-regulated compared to a reference expression profile consisting of the matching genes. “Similar” also includes a comparison in which two or more of the genes, including 3, 4, 5, 6, 7, . . . or all of the genes, from Tables 1, 2, and 3 in a subject's expression profile are up- or down-regulated, as are the matching genes in the reference expression profile.

An expression profile of one or more genes can be determined by microarray. Therefore, provided herein is an array comprising a substrate having a plurality of addresses, wherein each address comprises a capture probe that specifically binds under stringent conditions a nucleic acid corresponding to one or more of the DNA repair genes or other genes taught herein or any combination of the DNA repair genes or other genes taught herein or to a complement of those nucleic acids. A nucleic acid bound by the capture probe of each address is unique among the plurality of addresses. By “corresponding to” a nucleic acid of a gene is meant a nucleic acid that is a fragment of the gene such that the probe binds the gene or the complement thereof or binds the RNA or cDNA of the gene or fragments of the RNA or cDNA.

As used herein, “stringent conditions” refers to the hybridization and washing conditions used in a hybridization protocol. In general, the washing conditions should be a combination of temperature and salt concentration chosen so that the denaturation temperature is approximately 5-20° C. below the calculated Tm of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tm of such an oligonucleotide can be estimated by allowing 2° C. for each A or T nucleotide, and 4° C. for each G or C. For example, an 18 nucleotide probe of 50% G+C would, therefore, have an approximate Tm of 54° C. Stringent conditions are known to one of skill in the art. See, for example, Sambrook et al. (2001). An example of stringent wash conditions is 4× SSC at 65° C. Highly stringent wash conditions include, for example, 0.2× SSC at 65° C.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences of a plurality of genes taught herein. The substrate can be any substrate to which polynucleotide probes can be attached including, but not limited to, glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Tissue samples can be treated to form single-stranded polynucleotides, for example, by heating or by chemical denaturation, as is known in the art. The single-stranded polynucleotides in the tissue sample can then be labeled and hybridized to the polynucleotide probes on the array. Detectable labels which can be used include, but are not limited to, radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance. See Example 1 below.

Also provided is a method of identifying a tumor sensitive to a chemotherapeutic agent, comprising: a) determining an expression profile of one or more DNA repair genes or other genes taught herein from a tumor cell; and b) comparing the profile of step (a) to a reference expression profile, wherein the reference expression profile correlates with resistance to the chemotherapeutic agent, a dissimilar expression profile of genes from the tumor cell compared to the reference expression profile indicating sensitivity of the tumor cell to the chemotherapeutic agent, thereby identifying a tumor that is sensitive to the chemotherapeutic agent.

As used herein, “dissimilar” means “not resembling, not completely identical, or not at all alike.” Thus, a subject's expression profile can be dissimilar to a reference expression profile when, for example, the subject's expression profile contains all of the genes listed in Tables 1, 2, and 3 and none of those genes are up- or down-regulated compared to a reference expression profile consisting of the matching genes. “Dissimilar” also includes a comparison in which two or more of the genes, including 3, 4, 5, 6, 7, . . . or all of the genes, from Tables 1, 2, and 3 in a subject's expression profile are not up- or down-regulated, as are the matching genes in the reference expression profile.

Another aspect of the invention is a method of selecting a chemotherapeutic agent for treating a subject with a tumor, comprising: a) determining an expression profile of one or more DNA repair genes or other genes taught herein from a cell of the subject's tumor; b) comparing the expression profile of step (a) to a reference expression profile, wherein the reference expression profile correlates with resistance to the chemotherapeutic agent; and c) selecting the chemotherapeutic agent for treating the subject when the subject's expression profile is dissimilar to the reference expression profile.

By comparing a subject's tumor cell expression profile to a reference expression profile and finding that the two profiles are dissimilar, a person of skill can choose to treat the subject's tumor with the chemotherapeutic agent because the subject's tumor is not resistant (i.e., is sensitive) to the chemotherapeutic agent. It is within the scope of the invention to monitor the effectiveness of treatment of a subject's tumor with a selected chemotherapeutic agent to determine whether the tumor acquires resistance to the chemotherapeutic agent. Specifically, during the course of chemotherapy, a person of skill can sample cells from residual tumor and prepare an updated expression profile to compare to the reference expression profile that correlates with resistance to the selected chemotherapeutic. If during the course of treatment, the expression profile from the cells of the subject's residual tumor changes and becomes similar to the reference expression profile that correlates with resistance to the chemotherapeutic agent, it can be determined that the tumor has acquired resistance to the chemotherapeutic agent, and alternative therapies can be instituted. It follows that if the updated expression profile from the subject's residual tumor cells remains dissimilar to the reference expression profile, it can be determined that the tumor remains sensitive to the selected chemotherapeutic agent, and therapy can be continued.

Further provided is a method of selecting a chemotherapeutic agent for treating a subject with a tumor, comprising: a) isolating mRNA of a cell of the tumor; b) optionally converting the mRNA to cDNA by reverse-transcription; c) contacting the mRNA of step (a) and/or the cDNA of step (b) with an array comprising a substrate having a plurality of addresses, wherein each address comprises a capture probe that specifically binds under stringent conditions a nucleic acid corresponding to a DNA repair gene or other gene taught herein; d) detecting binding of the cDNA or mRNA to each address of the plurality of addresses to determine an expression profile; e) comparing the expression profile of step (d) with a reference expression profile, wherein the reference expression profile correlates with resistance to the chemotherapeutic agent and wherein a dissimilarity between the reference expression profile and the expression profile of step (d) indicates a chemotherapeutic agent to which the tumor is sensitive; and f) selecting the chemotherapeutic agent identified in step (e) for treating the subject.

Also provided is a computer-readable medium comprising a plurality of digitally encoded expression profiles, wherein each profile of the plurality has a plurality of values, each value representing the expression level of a DNA repair gene or other gene taught herein. Each profile of the plurality of digitally encoded expression profiles is associated with sensitivity to a chemotherapeutic agent.

Further provided is a kit for selecting a chemotherapeutic agent to treat a tumor in a subject, comprising: a) an array having a substrate including a plurality of addresses, wherein each address comprises a capture probe that specifically binds under stringent conditions a nucleic acid of a DNA repair gene or other gene taught herein or any combination thereof; and b) a computer-readable medium having a plurality of digitally encoded expression profiles, wherein each profile of the plurality has a plurality of values, each value representing the expression of a DNA repair gene or other gene taught herein and any combination thereof.

Also provided is a computer system comprising: a) a database having a plurality of digitally encoded reference expression profiles, wherein each profile of the plurality has (i) a plurality of values, each value representing the expression of a DNA repair gene or other gene taught herein or any combination thereof, and (ii) a pointer to a descriptor of a chemotherapeutic agent; and b) a server having computer-executable code for effecting the following steps: i) receiving a subject expression profile; ii) identifying from the database a matching reference expression profile that is most similar to the subject expression profile; and iii) outputting the descriptor to which the pointer of the matching reference profile points. The computer executable code further effects step iv) outputting a difference profile having a value representing the similarity of a value in the subject expression profile to a corresponding value in the matching reference profile.

It will be appreciated by those skilled in the art that the nucleic acids of the selected genes listed in Tables 1-3 as well as the nucleic acid sequences identified from subjects can be stored, recorded, and manipulated on any medium which can be read and accessed by a computer. As used herein, the words “recorded” and “stored” refer to a process for storing information on a computer medium. A person of skill can readily adopt any of the presently known methods for recording information on a computer readable medium to generate a list of sequences comprising one or more of the nucleic acids of the invention. Another aspect of the present invention is a computer readable medium having recorded thereon at least 2, 5, 10, 15, 20, 25, 30, 50, 100, 200, 250, 300, 400, 500, 1000, 2000, 3000, 4000 or 5000 nucleic acids of the invention or nucleic acid sequences identified from subjects.

Computer readable media include magnetically readable media, optically readable media, electronically readable media and magnetic/optical media. For example, the computer readable media may be a hard disc, a floppy disc, a magnetic tape, CD-ROM, DVD, RAM, or ROM as well as other types of other media known to those skilled in the art.

Embodiments of the present invention include systems, particularly computer systems, which contain the sequence information described herein. As used herein, “a computer system” refers to the hardware components, software components, and data storage components used to store and/or analyze the nucleotide sequences of the present invention or other sequences. The computer system preferably includes the computer readable media described above, and a processor for accessing and manipulating the sequence data.

Preferably, the computer is a general purpose system that comprises a central processing unit (CPU), one or more data storage components for storing data, and one or more data retrieving devices for retrieving the data stored on the data storage components. A person of skill can readily appreciate that any one of the currently available computer systems are suitable.

In one aspect, the computer system includes a processor connected to a bus which is connected to a main memory, preferably implemented as RAM, and one or more data storage devices, such as a hard drive and/or other computer readable media having data recorded thereon. In some aspects, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In some embodiments, the data storage component is a removable computer readable medium such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device. Software for accessing and processing the nucleotide sequences of the nucleic acids of the invention (such as search tools, compare tools, modeling tools, etc.) may reside in main memory during execution.

Further provided is a method of identifying a chemotherapeutic agent to which a subject's tumor is chemoresistant, comprising: a) providing a nucleic acid corresponding to one or more DNA repair genes or other genes taught herein or any combination thereof from the subject's tumor; b) preparing a subject expression profile from the nucleic acid; c) selecting from a plurality of reference expression profiles a matching reference expression profile most similar to the subject expression profile, wherein the reference expression profiles and the subject expression profile have a plurality of values, each value representing the expression level of a specific DNA repair gene or other gene taught herein or any combination thereof, and wherein each reference expression profile of the plurality of reference expression profiles correlates with resistance to a chemotherapeutic agent; and d) transmitting a descriptor of the chemotherapeutic agent associated with the matching reference expression profile, thereby identifying a chemotherapeutic agent to which the tumor is resistant.

Also provided is a method of identifying a chemotherapeutic agent to which a subject's tumor is sensitive, comprising: a) providing a nucleic acid corresponding to one or more DNA repair genes or other genes taught herein or any combination thereof from the subject's tumor; b) preparing a subject expression profile from the nucleic acid; c) selecting from a plurality of reference expression profiles a reference expression profile dissimilar to the subject expression profile, wherein the reference expression profiles and the subject expression profile have a plurality of values, each value representing the expression level of a specific DNA repair gene or other gene taught herein or any combination thereof, and wherein each reference expression profile of the plurality of reference expression profiles correlates with resistance to a chemotherapeutic agent; and d) transmitting a descriptor of the chemotherapeutic agent associated with the dissimilar reference profile, thereby identifying a chemotherapeutic agent to which the subject's tumor is sensitive.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Affymetrix® Microarray Analysis. Affymetrix® Human Genome 133A arrays were used to study the gene expression profiles of SW948, SW948CPTH, and SW948CPTL cell lines. The SW948CPTH cells were grown in the presence of 20 μg/ml CPT-11 for at least about 62 days followed by the removal of the drug. SW948CPTL cells were grown in the presence of 40 μg/ml CPT-11 for at least about 22 days or at least about 42 days followed by the removal of the drug. The SW948CPTH cells were collected for isolation of total RNA after removal of CPT-11 for 22 d, 32 d, 48 d, 65 d, 146 d and 175 d. The SW948CPTL cells were collected for isolation of total RNA after removal of CPT-11 for 28 d, 55 d and 175 d. SW948 cells were collected and processed at the same time as SW948CPTH and SW948CPTL cells. For all cell lines, the cells were collected from cultures that were 50-70% confluent and total RNA was isolated from 5-10×10⁶ cells with TRIzol reagent according to the manufacturer's instructions (Invitrogen Life Technology, Carlsbad, Calif.). cDNA was generated by linear amplification of the RNA using an oligo dT-T7 primer and reverse transcriptase. Subsequently, biotin labeled complementary RNA (cRNA) was synthesized by in vitro transcription (IVT) and then broken into 50-200 base fragments for more uniform hybridization kinetics. Prior to hybridizing to the U133A arrays, Affymetrix® test arrays were used to determine the quality of the hybridization target. RNA sample preparation, array hybridization, array washing, and scanning were performed following the protocols in Affymetrix® GeneChip Expression Analysis Technical manual. Initial data extraction, paired comparisons for fold change determination and data filtration were performed using Affymetrix Microarray Suite (5.1).

Complete analysis and filtration of the data were performed using GeneTraffic® (Iobion Informatics LLC, La Jolla, Calif.) and final comparisons and tables were created using Microsoft Office Access 2003® (Microsoft Corporation, Redmond, Wash.). Each of Tables 1-3 lists genes that were significantly up—(>1.0) or down—(<1.0) regulated as compared to the parental cell line, SW948.

For example, a combination of 5 independent microarray chip analyses has identified 11 DNA repair genes that are significantly up or down regulated in the SW948CPTH cell line as compared to the parental cell line, SW948 (Table 1). In another example, a combination of 3 independent microarray chip analyses has identified 4 informative DNA repair genes that are significantly up or down regulated in the SW948CPTL cell line as compared to the parental cell line, SW948 (Table 2). Combining the data from Tables 1 and 2 gives a total of 14 human DNA repair genes that are differentially expressed in the CPT-11 drug resistant cell lines as compared to the parental cell line, SW948.

### Example 2

Total RNA was isolated from frozen tissues using Trizol reagent (Invitrogen, Carlsbad, Calif.) with subsequent chloroform extraction. RNA was then DNase-treated and purified using the RNeasy Mini Kit (QIAGEN, Hilden, Germany) as per manufacturer's instructions. Total RNA was quantified spectrophotometrically by A260 measurement and diluted to a final concentration of 4 ng/μl in RNAse-free water containing 12.5 ng/μl of total yeast RNA (Ambion, Austin, Tex.) as a carrier. cDNA was prepared using the High Capacity cDNA Archive Kit (Applied Biosystems, Foster City, Calif.) as per manufacturer's instructions. The resulting cDNA samples were stored at −80° C. or used immediately for low density array (LDA) analysis.

cDNA was added to an equal volume of 2× Taqman Universal PCR Master Mix (Applied Biosystem, Foster City, Calif.). After gentle mixing and centrifugation, each cDNA sample was loaded onto a user-designed LDA (Applied Biosystems, Foster City, Calif.) using the loading ports at the top of the array. There are 8 loading ports on each array and each one connects to 48 separate wells for a total of 384 wells per array. Each well contains pre-designed primer and probe sets for a specific, user-defined gene. Each array was configured into 4 replicates of 96 genes. The 18S & RPLP0 (ribosomal protein, large, P0) housekeeping genes were included in the 96 genes to normalize for RNA concentration. The 94 genes included on each LDA were segregated into 5 different cards, each card containing genes associated with different cellular processes including anti-apoptotic, pro-apoptotic, angiogenic, DNA repair, methylation, ubiquitination, transcription factor, drug metabolism and kinase gene functional groups.

Expression values for all 384 genes were calculated using the comparative Ct method using the formula 2-ΔΔCt as previously described (1). RPLP0 was set as the endogenous reference gene to normalize for differences in RNA concentration between samples. Gene expression values for all resistant SW948CPTH and SW948CPTL samples were calculated relative to their corresponding sensitive (wild type SW948) cell line, which was assigned a normalized value of 1.00 for each gene. This microarray method confirmed the DNA chip array analysis and disclosed additional informative genes which are significantly differentially expressed in both the SW948CPTH and the SW948CPTL cell lines when compared to the wild-type SW948 cell line (Table 3).

TABLE 3
LOW DENSITY ARRAY ANALYSIS

| Gene Name (Gene) | Accession # |
|---|---|
| ATP7A | NM_000052 |
| dihydropyrimidinase-like 3 (DPYSL3) | NM_001387 |
| hypoxia-inducible factor 1, alpha (HIF1A, MOP1) | NM_001530 |
| met proto-oncogene (MET) | NM_000245 |
| metallothionein 1F (MT1F) | NM_005949 |
| transforming growth factor, beta receptor III (TGFBR3) | NM_003243 |
| cyclin D2 (CCND2) | NM_001759 |
| ERCC1 | NM_001983 |
| ERCC2 (XPD) | NM_000400 |
| ERCC6 | NM_000124 |
| mutL homolog 3 (MLH3) | NM_000463 |
| matrix metalloproteinase 7 (MMP7) | NM_002423 |
| sprouty homolog 4 (SPRY4) | NM_030964 |
| XRCC1 | NM_006297 |

The group of genes from the SW948CPTH and SW948CPTL cell lines that were found to be up/down regulated by both disclosed microarray methods include the following genes: XRCC1; MGMT; RPA2; CCNH; ERCC1; MUS81; XRCC5 (Ku80); DUT; REV1L (REV1); UBE2B (RAD6B); RECQL (RECQ1); PMS2; PCNA; TREX1 (DNase III); cyclin D2 (CCND2); ERCC1; ERCC2 (XPD); ERCC6; mutL homolog 3 (MLH3); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor, and beta receptor III (TGFBR3).

### Example 3

Determination of cross-resistance was determined by cell proliferation assay. SW948, SW948CPTH or SW948CPTL cells were plated in 24-well tissue culture plates and allowed to grow for 24 h at which time the drugs were added at various doses ranging from 0-80 μg/ml. Then 24 h later, the culture medium was removed, and the cells were washed once with PBS followed by the addition of drug-free culture medium. Cells were counted on day 4 using a cell counter (Beckman Coulter, Fullerton, Calif.). The data were analyzed with Microsoft Excel (Microsoft Corp., Redmond, Wash.) using an exponential regression model to determine the IC50 dose.

SW948CPTH cells were shown to be cross-resistant to camptothecin-related drugs, CPT-11, CPT, 10-OH-CPT and SN38. SW948CPTH cells were not cross-resistant to another Topoisomerase I inhibitor, labachone, and the topoisomerase II inhibitor, etoposide. No resistant was found by SW948CPTH cells to cisplatin, gemcitabine, doxorubicin and 5-FU. SW948CPTL cells showed resistance to CPT-11, CPT, SN38, etoposide, cisplatin, gemcitabine. SW948CPTL showed no resistance to 5-FU. The cross-resistance data in Table 4 showed a broad resistance to other drugs by SW948CPTL cells, whereas SW948CPTH showed a specific drug-resistance to camptothecin-related drugs.

TABLE 4
Cell proliferation assay used to examine the sensitivity of SW948, SW948CPTH,
and SW948CPTL to various drugs. The SW948CPTH cells were treated with 20
μg/ml CPT-11 for 62 days, then the drug was removed and cross-resistance
studies were performed on cells without exposure to CPT-11 for greater than
22 days. The SW948CPTL cells were treated with 40 μg/ml CPT-11 for 42
days, then the drug was removed and cross-resistance studies were performed
on cells without exposure to CPT-11 for greater than 28 days. Results are
expressed as mean IC₅₀ dose (μg/ml) ± SD from 1-4 independent assays
done in quadruplicate. Significant change in resistance compared to the SW948
cell line was determined by standard t-test.

| | | SW948CPTH | SW948CPTL |
|---|---|---|---|
| Drug (μg/ml) | SW948 | (fold change) | (fold change) |
| CPT-11 | 3.0 ± 0.3 | 38 ± 6.9 (12)* | 15.2 ± 2 (5)* |
| CPT | 0.057 ± 0.02 | 0.32 ± 0.02 (6)* | 0.14 (2.4) |
| 10-OH-CPT | 0.25 ± 0.16 | 1.7 ± 0.3 (7)* | ND¹ |
| SN38 | 0.19 ± 0.01 | 1.5 ± 0.13 (8)* | 1.7 ± 0.15 (9)* |
| Lapachone | 1.06 ± 0.16 | 0.84 ± 0.21 | ND |
| Etoposide | 0.8 ± 0.1 | 0.7 ± 0.2 | 1.6 ± 0.2 (2)* |
| Cisplatin | 1.3 ± 0.34 | 1.5 ± 0.15 | 2.2 ± 1.3 (1.7) |
| Gemcitabine | 0.009 ± 0.004 | 0.008 ± .004 | 0.016 (1.8) |
| Doxorubicin | 0.037 ± 0.008 | 0.048 ± 0.008 | ND |
| 5-FU | 0.61 ± 0.1 | 0.54 ± 0.2 | 0.75 ± 0.02 |

*p-value < 0.05,
¹ND = not determined

### Example 4

This experiment shows reduction of drug resistance caused by decreasing XRCC1 protein in SW948CPTH cells using cetuximab (C225) or small inhibitory RNAs to XRCC1 (siXRCC1). Standard cell proliferation assays were done in 24-well tissue culture plates. After plating the cells in the plates, C225 (5 μg/ml), oligofectamine or siXRCC1 (60 pmoles)+oligofectamine was added to 1 ml culture medium per well. Following a 24 h incubation at 37° C., the cell culture media from the oligo and siXRCC1 groups were removed; the cells were washed with 1 ml PBS, and culture medium was added to the cells. Serial dilutions of CPT-11 were added to each well in quadruplicate. After a 24 h incubation at 37° C., the culture medium was removed from all groups, the cells washed with PBS and drug-free medium added to the cells. C225 (5 μg/ml) was added to the culture medium in the C225 treated group. Cells were counted 96 h after CPT-11 treatment. IC50 doses for CPT-11 were calculated by fitting the data to an exponential regression model (Microsoft Excel, Microsoft Corp., Redmond, Wash.).

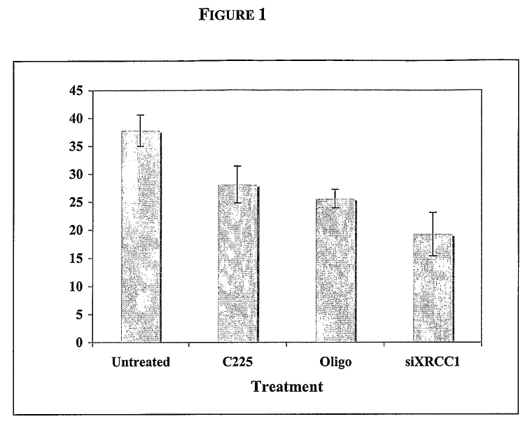

A significant decrease, 26% and 25%, was observed after treatment of the SW948CPTH cells with C225 and siXRCC1, respectively. Oligofectamine decreased the IC50 dose for SW948CPTH cells because the liposomal structure of oligofectamine increased the uptake of CPT-11 (FIG. 1).

### Example 5

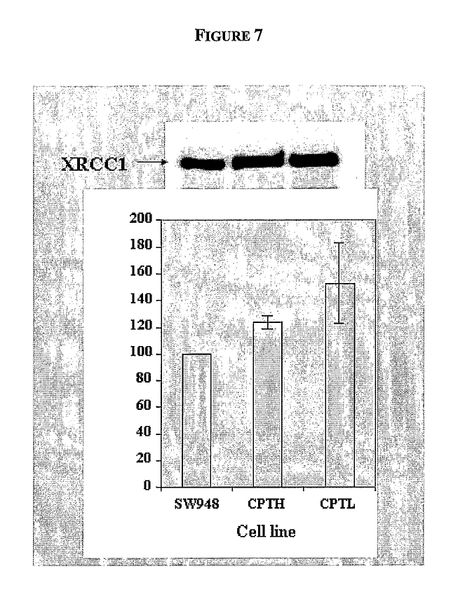

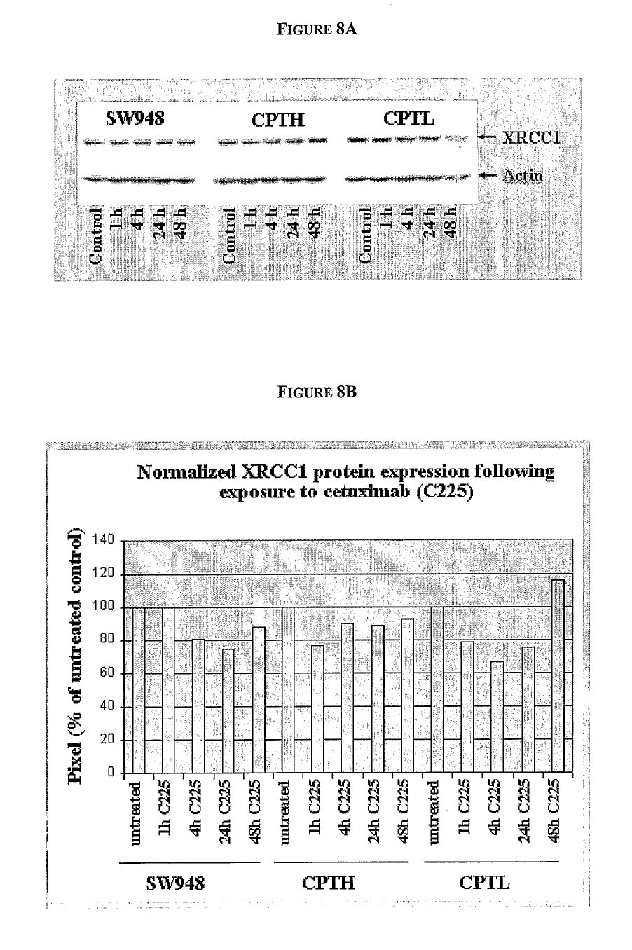

This experiment shows an immunoblot of XRCC1 protein from SW948, SW948CPTH cells exposed to cetuximab (IMC-C225) for 0 h, 4 h, 24 h, 48 h and 72 h. Cetuximab was added at time 0 at a concentration of 5 μg/ml. The cells were incubated at 37° C., and cell lysates were collected at each time point. For the detection of XRCC1, 20 μg protein was loaded per lane on a 10% SDS-PAGE. The proteins were separated by electrophoresis and then transferred to a nylon membrane. The blot was probed with anti-XRCC1 (NeoMarkers) followed by anti-mouse-HRP. The blots were developed with chemiluminescence and scanned using UN-SCAN-IT software (FIGS. 7 and 8).

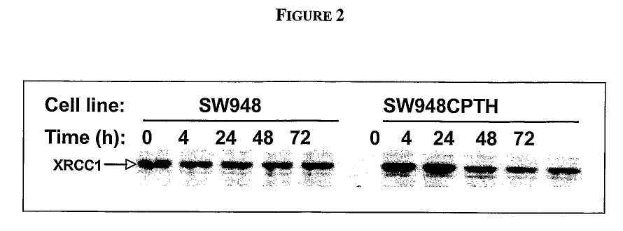

Cetuximab reduced the expression of XRCC1 after 24 h incubation (FIG. 2). The decreased expression of XRCC1 was maintained for 72 h. Cetuximab showed no change in XRCC1 protein expression in SW948 cells. Cetuximab's ability to reduce XRCC1 expression in SW948CPTH cells can be all or part of the mechanism for the increased sensitivity of SW948CPTH cells to CPT-11 treatment.

### Example 6

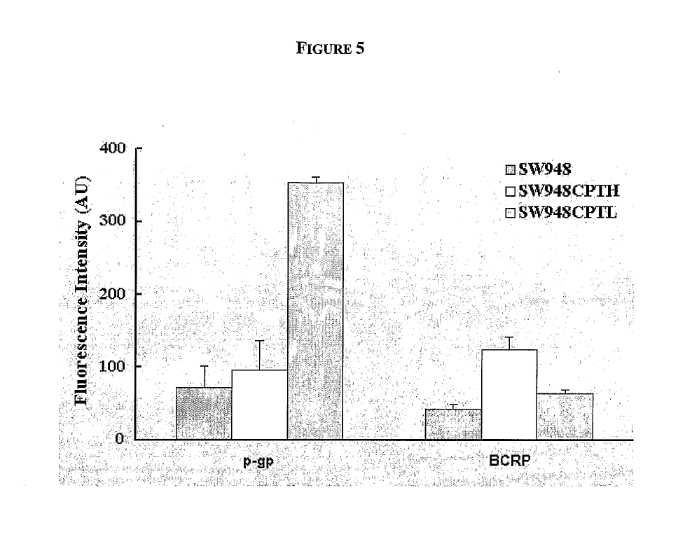

SW948CPTH and SW948CPTL cells were cultured in medium without CPT-11 for 21 or 7 days, respectively. Cells were washed with phosphate buffered saline, scraped, and aliquoted into tubes. Cells were incubated with antibodies against p-gp or BCRP for 30 min at 4° C., followed by FITC-conjugated goat anti-mouse antibody. Fluorescence intensity of the cells was monitored and analyzed by flow cytometry. Data are shown as mean fluorescence intensity ±SD (n=3-6). See FIG. 5.

### Example 7

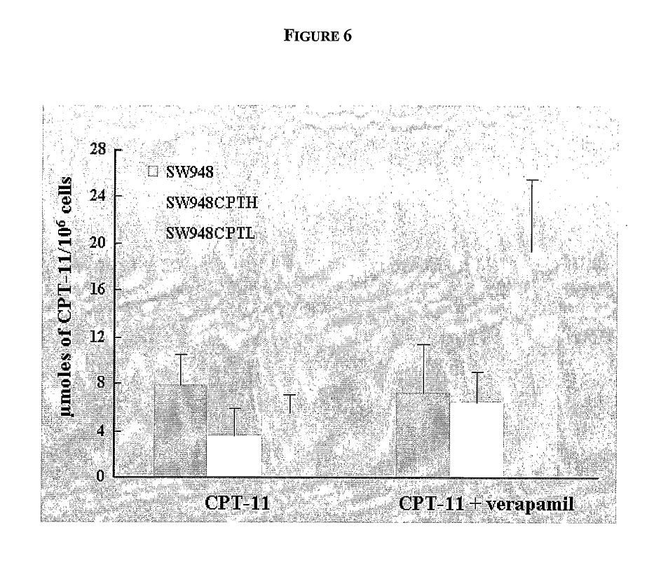

This experiment shows the effects of verapamil on the intra-cellular accumulation of CPT-11 in SW948, SW948CPTH, and SW948CPTL cells. Cells were incubated with CPT-11 [160 μM] for 2 h at 37° C. in the presence or absence of 10 μM verapamil. Cells were washed, trypsinized, and counted. Cell pellets were lysed with water and sonicated. The amounts of CPT-11 in the supernatant harvested from the samples were determined by HPLC. The values of CPT-11 were calculated as μmoles/106 cells. Data are expressed as mean ±SD (n=3). See FIG. 6.

### Example 8

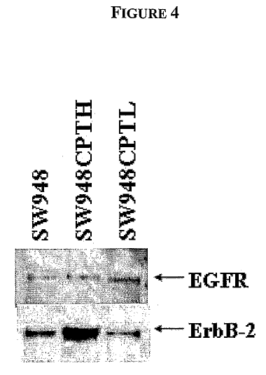

Expression of EGFR and ErbB2 protein from SW948, SW948CPTH, and SW948CPTL were measured from cell lysates. 20 μg protein was loaded per lane. The blots were probed with anti-EGFR (Sigma) or ErbB-2 (Santa Cruz), followed by anti-mouse-HRP or anti-rabbit-HRP, respectively. The blots were developed with chemiluminescence (FIG. 4).

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Johnson M R, Wang K, Smith J B, Heslin M J, Diasio R B. Quantitation of dihydropyrimidine dehydrogenase expression by real-time reverse transcription polymerase chain reaction. Anal Biochem. 2000 Feb. 15, 278(2): 175-84.
2. PCT/US04/29696, filed Sep. 10, 2004.

1. A method of determining resistance of a tumor cell to a chemotherapeutic agent, comprising:
a) determining an expression profile of one or more genes from the tumor cell, wherein the genes are DNA repair genes; and
b) comparing the profile of step a) to a reference expression profile, wherein the reference profile correlates with resistance to the chemotherapeutic agent, a similar expression profile of genes from the tumor cell compared to the reference expression profile indicating resistance of the tumor cell to the chemotherapeutic agent.

2. The method of claim 1, wherein the DNA repair genes are selected from the group of genes consisting of XRCC1, MGMT, RPA2, CCNH, ERCC1, MUS81, XRCC5 (Ku80), DUT, REV1L (REV1), UBE2B (RAD6B), RECQL (RECQ1), ERCC2 (XPD), ERCC6, PMS2, PCNA, TREX1 (DNase III) and mutL homolog 3 (MLH3).

3. The method of claim 1, wherein the chemotherapeutic agent is camptothecin (CPT) or a derivative or a metabolite thereof.

4. The method of claim 3, wherein the derivative of CPT is CPT-11 or 10-OH-CPT.

5. The method of claim 3, wherein the metabolite of CPT-11 is SN38.

6. The method of claim 1, wherein the chemotherapeutic agent is selected from the group consisting of actinomycin D, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

7. A method of determining resistance of a tumor cell to a chemotherapeutic agent, comprising:
a) determining an expression profile of one or more genes from the tumor cell, wherein the genes are selected from the group consisting of cyclin D2 (CCND2); matrix metalloproteinase 7 (MMP7); sprouty homolog 4 (SPRY4); ATP7A, dihydropyrimidinase-like 3 (DPYSL3), hypoxia-inducible factor 1, alpha (HIF1A, MOP1); met proto-oncogene (MET); metallothionein 1F (MT1F); and transforming growth factor beta receptor III (TGFBR3); and
b) comparing the profile of step a) to a reference expression profile, wherein the reference profile correlates with resistance to the chemotherapeutic agent, a similar expression profile of genies from the tumor cell compared to the reference expression profile indicating resistance of the tumor cell to the chemotherapeutic agent.

8. The method of claim 7, wherein the chemotherapeutic agent is camptothecin (CPT) or a derivative or a metabolite thereof.

9. The method of claim 8, wherein the derivative of CPT is CPT-11 of 10-OH-CPT.

10. The method of claim 8, wherein the metabolite of CTP-11 is SN38.

11. The method of claim 7, wherein the chemotherapeutic agent is selected from the group consisting of actinomycin D, capecitabine, carboplatin, cisplatin, colchicine, daunorubicin, docetaxel, doxorubicin, epirubicin, etoposide, 5-fluorouracil, gemcitabine, melphalan, methotrexate, mitomycin C, mitoxantrone, paclitaxel, topotecan, vinblastine, and vincristine.

12. A method of identifying a tumor sensitive to a chemotherapeutic agent, comprising:
a) determining an expression profile of one or more genes from the tumor cell, wherein the genes are DNA repair genes; and
b) comparing the profile of step a) to a reference expression profile, wherein the reference profile correlates with resistance to the chemotherapeutic agent, a dissimilar expression profile of genes from the tumor cell compared to the reference expression profile indicating sensitivity of the tumor cell to the chemotherapeutic agent, thereby identifying a tumor that is sensitive to the chemotherapeutic agent.

13. The method of claim 12, wherein the DNA repair genes are selected from the group of genes consisting of XRCC1, MGMT, RPA2, CCNH, ERCC1, MUS81, XRCC5 (Ku80), DUT, REV1L (REV1), UBE2B (RAD6B), RECQL (RECQ1), ERCC2 (XPD), ERCC6, PMS2, PCNA, TREX1 (DNase 111) and mutL homolog 3 (MLH3).

14. A method of identifying a tumor sensitive to a chemotherapeutic agent, comprising:
a) determining an expression profile of one or more genes from the tumor cell, wherein the genes are selected from the group consisting of cyclin D2 (CCND2); matrix metalloproteinase 7 (MMP7); sprouty, homolog 4 (SPRY4); ATP7A, dihydropyrimidinase-like 3 (DPYSL3); hypoxia-inducible factor 1, alpha (HIF1A, MOP1) met proto-oncogene (MET); metallothionein 1F (Mt1F); and transforming growth factor, beta receptor III (TGFBR3); and
b) comparing the profile of step a) to a reference expression profile, wherein the reference profile correlates with resistance to the chemotherapeutic agent, a dissimilar expression profile of genes from the tumor cell compared to the reference expression profile indicating sensitivity of the tumor cell to the chemotherapeutic agent, thereby identifying a tumor that is sensitive to the chemotherapeutic agent.

15. A method of selecting a chemotherapeutic agent for treating a subject with a tumor, comprising:
a) determining an expression profile of one or more genes from the tumor cell, wherein the genes are DNA repair genes;
b) comparing the expression profile of step a) to a reference expression profile, wherein the reference expression profile correlates with resistance to the chemotherapeutic agent; and
c) selecting the chemotherapeutic agent for treating the subject when the subject's expression profile is dissimilar to the reference expression profile.

16. The method of claim 15, wherein the DNA repair genes are selected from the group of genes consisting of XRCC1, MGMT, RPA2, CCNH, ERCC1, MUS81, XRCC5 (Ku80), DUT, REV1L (REV1), UBE2B (RAD6B), RECQL, (RECQ1), ERCC2 (XPD), ERCC6, PMS2, PCNA, TREX1 (DNase III) and mutL homolog 3 (MLH3).

17. A method of selecting a chemotherapeutic agent for treating a subject with a tumor, comprising:
a) determining an expression profile of one or more genes from the tumor cell, wherein the genes are selected from the group consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha HIF1A, MOP1); met proto-oncogene (MET); metallothionein 1F (MT1F); and transforming growth factor, beta receptor III (TGFBR3);
b) comparing the expression profile of step a) to a reference expression profile wherein the reference expression profile correlates with resistance to the chemotherapeutic agent; and
c) selecting the chemotherapeutic agent for treating the subject when the subject's expression profile is dissimilar to the reference expression profile.

18. An array comprising a substrate having a plurality of addresses, wherein each address consists of a capture probe that specifically binds under stringent conditions a nucleic acid corresponding to the genes XRCC1; MGMT; RPA2; CCNH; ERCC1; MUS81; XRCC5 (Ku80); DUT; REV1L (REV1); UBE2B (RAD6B); RECQL (RECQ1); PMS2; PCNA; TREX1 (DNase III); cyclin D2 (CCND2); ERCC1; ERCC2 (XPD); ERCC6; mutL homolog 3 (MLH3); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor beta receptor III (TGFBR3).

19. A method of selecting a chemotherapeutic agent for treating a subject with a tumor, comprising:
a) isolating mRNA of a cell of the tumor;
b) optionally converting the mRNA to cDNA by reverse-transcription;
c) contacting the mRNA of step (a) or the cDNA of step (b) with the array of claim 18;
d) detecting binding of the cDNA or mRNA to each address of the plurality of addresses to determine an expression profile;
e) comparing the expression profile of step c) with a reference expression profile, wherein the reference expression profile correlates with resistance to the chemotherapeutic agent and wherein a dissimilarity between the reference expression profile and the expression profile of step c) indicates a chemotherapeutic agent to which the tumor is sensitive; and
f) selecting the chemotherapeutic agent identified in step (e) for treating the subject.

20. A computer-readable medium comprising a plurality of digitally encoded expression profiles, wherein each profile of the plurality has a plurality of values, each value representing the expression level of a DNA repair gene.

21. The computer-readable medium of claim 20, wherein the DNA repair genes are selected from the group of genes consisting of XRCC1, MGMT, RPA2, CCNH, ERCC1, MUS81, XRCC5 (Ku80), DUT, REV1L (REV1), UBE2B (RAD6B), RECQ1 (RECQ1) ERCC2 (XPD), ERCC6, PMS2, PCNA, TREX1 (DNase III) and mutL homolog 3 (MLH3).

22. The computer-readable medium of claim 20, wherein each profile of the plurality of digitally encoded expression profiles is associated with a chemotherapeutic agent.

23. A computer-readable medium comprising a plurality of digitally encoded expression profiles, wherein each profile of the plurality has a plurality of values, each value representing the expression level of a gene selected from the group of genes consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor, beta receptor III (TGFBR3).

24. The computer-readable medium of claim 23, wherein each profile of the plurality of digitally encoded expression profiles is associated with a chemotherapeutic agent.

25. A kit for selecting a chemotherapeutic agent to treat a tumor in a subject, comprising:
a) an array having a substrate including a plurality of addresses, wherein each address consists of a capture probe that specifically binds under stringent conditions a nucleic acid of one DNA repair gene; and
b) a computer-readable medium having a plurality of digitally encoded expression profiles, wherein each profile of the plurality has a plurality of values, each value representing the expression of one DNA repair gene.

26. The kit of claim 20, wherein the DNA repair gene is selected from the group of genes consisting of XRCC1, MGMT, RPA2, CCNH, ERCC1, MUS81, XRCC5 (Ku80), DUT, REV1L (REV1), UBE2B (RAD6B), RECQL (RECQ1), ERCC2 (XPD), ERCC6, PMS2, PCNA, TREX1 (DNase III) and mutL homolog 3 (MLH3).

27. A kit for selecting a chemotherapeutic agent to treat a tumor in a subject, comprising:
a) an array having a substrate including a plurality of addresses, wherein each address consists of a capture probe that specifically binds under stringent conditions a nucleic acid of one gene selected from the group of genes consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor, beta receptor III (TGFBR3),
b) a computer-readable medium having a plurality of digitally encoded expression profiles, wherein each profile of the plurality has a plurality of values, each value representing the expression of one gene selected from the group of genes consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor, beta receptor III (TGFBR3).

28. A computer system comprising:
a) a database having a plurality of digitally, encoded reference expression profiles, wherein each profile of the plurality has (i) a plurality of values, each value representing the expression of a DNA repair gene, (ii) a pointer to a descriptor of a chemotherapeutic; and
b) a server having computer-executable code for effecting the following steps
i) receiving a subject expression profile.
ii) identifying from the database a matching reference expression profile that is most similar to the subject expression profile; and
iii) outputting the descriptor to which the pointer of the matching reference profile points.

29. The computer system of claim 28, wherein the DNA repair gene is selected from the group of genes consisting of XRCC1, MGMT, RPA2, CCNH, ERCC1, MUS81, XRCC5 (Ku80), DUT, REV1L, (REV1), UBE2B (RAD6B), RECQL (RECQ1), ERCC2 (XPD), ERCC6, PMS2, PCNA, TREX1 (DNase III) and mutL homolog 3 (MLH3).

30. The computer system of claim 28, wherein the computer executable code further effects step iv) outputting a difference profile having a value representing the similarity of a value in the subject expression profile to a corresponding value in the matching reference profile.

31. A computer system comprising;
a) a database having a plurality of digitally encoded reference expression profiles, wherein each profile of the plurality has (i) a plurality of values, each value representing, the expression of a gene selected from the group of genes consisting of cyclin D2 (CCND2); matrix metalloproteinase 7 (MMP7); sprouty homolog 4 (SPRY4); ATP7A, dihydropyrimidinase-like 3 (DPYSL3) hypoxia-inducible factor 1, alpha (HIF1A, MOP1); met proto-oncogene (MET); metallothionein 1F (MF1F); and transforming growth factor, beta receptor III (TGFBR3), and (ii) a pointer to a descriptor of a chemotherapeutic: and
b) a server having computer-executable code for effecting the following steps:
i) receiving a subject expression profile;
ii) identifying from the database a matching reference expression profile that is most similar to the subject expression profile; and
iii) outputting the descriptor to which the pointer of the matching reference profile points.

32. The computer system of claim 31, wherein the computer executable code further effects step iv) outputting a difference profile having a value representing the similarity of a value in the subject expression profile to a corresponding value in the matching reference profile.

33. A method of identifying a chemotherapeutic agent to which a subject's tumor is chemoresistant, comprising:
a) providing) a nucleic acid corresponding to one or more DNA repair genes from the subject's tumor;
b) preparing a subject expression profile from the nucleic acid;
c) selecting from a plurality of reference expression profiles a matching reference expression profile most similar to the subject expression profile, wherein the reference expression profiles and the subject expression profile have a plurality of values, each value representing the expression level of a specific DNA repair gene, and wherein each reference expression profile of the plurality of reference expression profiles correlates with resistance to a chemotherapeutic agent; and
d) transmitting a descriptor of the chemotherapeutic associated with the matching reference expression profile, thereby identifying a chemotherapeutic agent to which the tumor is resistant.

34. The method of claim 33, wherein the DNA repair gene is selected from the group of genes consisting of XRCC1, MGMT, RPA2, CCNH, ERCC1, MUS81, XRCC5 (Ku80), DUT, REV1L (REV1), UBE2B (RAD6B), RECQL (RECQ1), ERCC2) (XPD), ERCC6, PMS2, PCNA, TREX1 (DNase III) and mutL homolog 3 (MLH3).

35. A method of identifying a chemotherapeutic agent to which a subject's tumor is chemoresistant, comprising:
a) providing, a nucleic acid corresponding to one or more genes selected from the group of genes consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F), and transforming growth factor, beta receptor III (TGFBR3) from the subject's tumor;
b) preparing a subject expression profile from the nucleic acid;
c) selecting from a plurality of reference expression profiles a matching reference expression profile most similar to the subject expression profile, wherein the reference expression profiles and the subject expression profile have a plurality of values, each value representing the expression level of a specific DNA repair gene, and wherein each reference expression profile of the plurality of reference expression profiles correlates with resistance to a chemotherapeutic agent; and
d) transmitting a descriptor of the chemotherapeutic associated with the matching reference expression profile, thereby identifying a chemotherapeutic agent to which the tumor is resistant.

36. A method of identifying a chemotherapeutic agent to which a subject's tumor is sensitive, comprising;
a) providing a nucleic acid corresponding to one or more DNA repair genes from the subject's tumor;
b) preparing a subject expression profile from the nucleic acid;
c) selecting from a plurality of reference expression profiles a reference expression profile dissimilar to the subject expression profile, wherein the reference expression profiles and the subject expression profile have a plurality of values, each value representing, the expression level of a specific DNA repair gene, and wherein each reference expression profile of the plurality of reference expression profiles correlates with resistance to a chemotherapeutic agent; and
d) transmitting a descriptor of the chemotherapeutic agent associated with the dissimilar reference profile, thereby identifying a chemotherapeutic agent to which the tumor its sensitive.

37. The method of claim 36, wherein the DNA repair gene is selected from the group of genes consisting of XRCC1, MGMT, RPA2, CCNH, ERCC1, MUS81, XRCC5 (Ku80), DUT, REV1L (REV1), UBE2B (RAD6B), RECQL (RECQ1), ERCC2 (XPD), ERCC6, PMS2, PCNA, TREX1 (DNase III) and mutL homolog 3 (MLH3).

38. A method of identifying a chemotherapeutic agent to which a subject's tumor is sensitive, comprising:
a) providing, a nucleic acid corresponding to one or more genes selected from the group of genes consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor, beta receptor III (TGFBR3) from the subject's tumor;
b) preparing a subject expression profile from the nucleic acid;
c) selecting from a plurality of reference expression profiles a reference expression profile dissimilar to the subject expression profile, wherein the reference expression profiles and the subject expression profile have a plurality of values, each value representing the expression level of a specific gene selected from the group of genes consisting of cyclin D2 (CCND2); matrix metalloproteinase 7 (MMP7); sprouty homolog 4 (SPRY4); ATP7A, dihydropyrimidinase-like 3 (DPYSL3); hypoxia-inducible factor 1, alpha (HIF1A, MOP1); met proto-oncogene (MET); metallothionein 1F (MT1F); and transforming growth factor, beta receptor III (TGFBR3), and wherein each reference expression profile of the plurality of reference expression profiles correlates with resistance to a chemotherapeutic agent; and
d) transmitting a descriptor of the chemotherapeutic agent associated with the dissimilar reference profile, thereby identifying a chemotherapeutic agent to which the tumor is sensitive.
